(19) United States
(12) Patent Application Publication
(10) Pub. No.: US 2014/0271754 A1
(43) Pub. Date: Sep. 18, 2014

(54) POLYMERIC MATERIALS PROVIDING IMPROVED INFRARED EMISSIVITY

(71) Applicant: Clopay Plastic Products Company, Inc., Mason, OH (US)

(72) Inventors: Carol Blaney, Cincinnati, OH (US); Gregory K. Jones, Lebanon, OH (US); David G. Bland, Painesville, OH (US)

(73) Assignee: Clopay Plastic Products Company, Inc., Mason, OH (US)

(21) Appl. No.: 14/030,084

(22) Filed: Sep. 18, 2013

Related U.S. Application Data
(60) Provisional application No. 61/792,414, filed on Mar. 15, 2013.

(51) Int. Cl. A61K 41/00; A61K 9/70

(52) U.S. Cl. CPC A61K 41/00; A61K 9/70
USPC 424/402; 424/443; 424/125

(57) ABSTRACT

Polymeric fibers and films that incorporate IR-emitting materials are disclosed. These fibers and films can be loaded with higher concentrations of IR-emitting materials than was previously thought possible. The IR emissivity of the resulting polymeric materials can be enhanced by increasing the surface area of these fibers and films. Laminates of the fibers or films with other substrate layers are also disclosed.

### RELATED APPLICATION(S)

This application is related to and claims the benefit of U.S. Patent Application Ser. No. 61/792,414, filed on Mar. 15, 2013, the disclosure of which is incorporated herein by reference.

### FIELD OF INVENTION

The present invention relates to polymeric materials that provide improved infrared (IR) emissivity. The polymeric materials, including polymeric films and fibrous webs, comprise IR emitting additives which may provide health benefits to living tissue in contact with or near the polymeric materials.

### BACKGROUND OF THE INVENTION

Infrared (IR) radiation is emitted as radiant heat from all physical objects above absolute zero. The ideal example of an IR emitter is a perfect black body which neither reflects nor transmits energy impinging upon it, but rather absorbs all energy impinging upon it. The black body, at steady state, then re-emits this absorbed energy in frequencies dependant on the temperature of that black body. A black body has an emissivity of 100%, a reflectivity of 0%, and a transmissivity of 0%.

Emitted IR energy has been studied for apparent benefits to living tissue. IR appears to enhance vital biological activity, such as expanding microcapillaries to enhance blood flow, boosting blood oxygen levels, and improving nutrient transport in and out of cells.

There is interest in materials that are close to black bodies in that they can absorb ambient energy from sources such as sunlight, artificial light, body heat, other ambient heat, electromagnetic energy, etc. and then, at steady state, re-emit this energy. Of particular interest are materials that emit IR energy, particularly in the 4 to 14 micron wavelength range of the far infrared spectrum, which is thought to be especially beneficial. Such materials that emit energy in this way may be very beneficial in enhancing healthy biological activity in living tissue. There is evidence, for instance, that packaging materials containing these IR-emitting materials aid in maintaining and lengthening the freshness of foodstuffs such as fresh produce and meat products. There is also evidence that garments, medical devices, blankets, and other such durable goods may enhance the physical health and well-being of living creatures including humans. Such durable goods have been shown to reduce inflammation, enhance blood oxygenation and other natural health-inducing biological functions, and improve performance during physical exertion such as exercise and sports. For these reasons, IR emitting materials are sometimes described as being ‘bio-active.’

Suitable IR emitting materials come from many sources. Various minerals and inorganic materials have been shown to exhibit IR emitting properties. Some organic materials also have these properties. It has been found that the IR emitting materials work best when pulverized into fine powders including nano-sized particles. The powders can be applied to the surfaces of base materials, such as fabrics, films or other solid objects. The powders can also be mixed into coatings or paints and applied to the surfaces of solid objects. The powders can also be mixed into a liquid or molten matrix, such as a molten thermoplastic polymer, and then molded into useful objects.

However, these IR emitting materials have drawbacks. Many of them are typically very expensive. Therefore these materials are often used only in very high-end durable goods, because of their high cost. Also, these materials can be difficult to incorporate into consumer goods. As noted above, these materials are most effective when pulverized into fine powders. Incorporating such powders into consumer goods can present challenges. Mineral-based or inorganic IR emitting powders do not have a natural affinity for the organic materials used in most consumer goods. For garments, IR emitting powders can be sprayed onto the surface of natural fibers, such as cotton, linen, and wool, but these powders can be removed or lost due to abrasion during wear or when washing the garment. Incorporating IR emitting powders into synthetic polymers is feasible. But there is a limit to how much IR emitting powder can be blended into a thin polymer material like a fiber or thin film before the polymeric material can no longer be manufactured. For instance, it has been found that inorganic IR emitting powders can be successfully blended into polymeric fibers at concentrations of no more than about 1-3% before the fiber becomes too easily broken during manufacture. It is unclear if garments, drapes, blankets, bandages, and other such consumer goods containing such a low level of IR-emitting powders are as effective in providing the health benefits these materials promise. Higher concentrations of IR emitting powders also weaken extruded films.

Therefore, there is a continuing need to improve materials, particularly synthetic polymeric materials that incorporate IR-emitting powders in order to provide benefits to living tissues. Reducing the cost of these polymeric materials is highly desirable, as is improving the performance of the IR-emitting powders contained within the materials.

### SUMMARY OF THE INVENTION

In one embodiment, the present invention is directed to polymeric fibers that incorporate IR-emitting powders in a component of bicomponent or multicomponent fibers.

In another embodiment, the present invention is directed to polymeric fibers that incorporate IR-emitting powders in the sheath layer of sheath-core bicomponent or multicomponent fibers

In another embodiment, the present invention is directed to polymeric fibers that incorporate IR-emitting powders in the islands of islands-in-the-sea bicomponent or multicomponent fibers.

In another embodiment, the present invention is directed to polymeric fibers that incorporate IR-emitting powders where the polymeric fibers have an increased surface area.

In another embodiment, the present invention is directed to polymeric fibers that incorporate IR-emitting powders where the polymeric fibers have an increased surface area due to the cross-section shape of the fiber.

In another embodiment, the present invention is directed to polymeric fibers that incorporate IR-emitting powders where the polymeric fibers have an increased surface area that is microtextured.

In another embodiment, the present invention is directed to polymeric fibers that incorporate IR-emitting powders where the polymeric fibers have an increased surface area that is cracked.

In another embodiment, the present invention is directed to polymeric fibers that incorporate IR-emitting powders where the polymeric fibers have an increased surface area that is foamed.

In another embodiment, the present invention is directed to polymeric fibers that incorporate IR-emitting powders where the polymeric fibers have an increased surface area because the fiber is split into microfibers.

In another embodiment, the present invention is directed to multilayer polymeric films that incorporate IR-emitting powders in one or more layers of the multilayer film.

In another embodiment, the present invention is directed to multilayer polymeric films that incorporate IR-emitting powders in one or more surface layers of the multilayer film.

In another embodiment, the present invention is directed to polymeric films that incorporate IR-emitting powders where the films have increased surface area.

In another embodiment, the present invention is directed to polymeric films that incorporate IR-emitting powders where the films have an increased surface area that is microtextured.

In another embodiment, the present invention is directed to polymeric films that incorporate IR-emitting powders where the films have an increased surface area that is embossed.

In another embodiment, the present invention is directed to polymeric films that incorporate IR-emitting powders where the films have an increased surface area that is cracked.

In another embodiment, the present invention is directed to polymeric films that incorporate IR-emitting powders where the films have an increased surface area that is foamed.

Additional embodiments of the invention will be apparent in view of the following detailed description of the invention.

### BRIEF DESCRIPTION OF THE DRAWINGS

The invention will be more fully understood in view of the drawings, in which:

FIGS. 1a-1c are cross sectional views of common structures for bicomponent fibers.

FIGS. 2a-2e are cross sectional views of common non-circular fibers.

FIG. 3 is a cross sectional view of a trilobal bicomponent fiber with one component concentrated at the end of the lobes.

FIG. 4 is a perspective view of a sheath-core bicomponent fiber where the sheath is microtextured.

FIG. 5 is a perspective view of a sheath-core bicomponent fiber where the sheath is cracked.

FIGS. 6a and 6b are representations of two bicomponent fibers before and after they are split into microfibers.

FIGS. 7a and 7b are graphs showing the measured emissivity for a foamed and flat polymer film of the present invention.

### DETAILED DESCRIPTION

The inventors have discovered that the effectiveness of IR-emitting powders in polymeric materials can be improved in several ways. For instance, it has been discovered that higher loadings of IR-emitting powders can be incorporated into one component of bicomponent or multicomponent fibers. The filled component of the fiber provides the IR-emitting properties, while the unfilled component of the fiber strengthens and supports the overall structure of the fiber, particularly during the spinning process. This multicomponent or multilayered effect is also seen in polymeric films incorporating IR-emitting powders. The inventors have also discovered that increasing the surface area of the polymeric material, whether in fibrous or film form, can improve the emissivity of the material.

For the purpose of this disclosure, the following terms are defined:

“Film” refers to material in a sheet-like form where the dimensions of the material in the x (length) and y (width) directions are substantially larger than the dimension in the z (thickness) direction. Films have a z-direction thickness in the range of about 1 μm to about 1 mm.

“Fiber” refers to a material in a thread-like form where the dimension of the material in the x (length) direction is substantially larger than the dimension in the y (width) and z (thickness) directions. Fibers may be solid throughout, or they may contain hollow areas, such as cells, bubbles or tubes. Staple fibers may be relatively short, with a length (x dimension) of about 2-100 mm. Continuous fibers, also known as filaments, are continuously spun and may be essentially infinite in length. Fibers typically have y and z dimensions that are less than an order of magnitude different, and these dimensions are in the range of about 0.1 μm to about 1 mm. For instance, a typical fiber may be roughly circular in cross section, with a diameter (y and z dimensions) of about 5 μm; alternatively, a typical fiber with an oval or oblong cross-section might have a width (y dimension) of about 10 μm but a thickness (z direction) of about 2 μm.

“Bicomponent fiber” refers to a fiber that comprises two distinctly different polymeric components that are simultaneously extruded to form the fiber, but the components remain essentially separate and unmixed within the structure of the fiber. Bicomponent fibers are therefore somewhat comparable to coextruded multilayer polymer films. Typical cross-sectional structures of bicomponent fibers include sheath/core, side-by-side, islands-in-the-sea, pie or orange structures.

“Multicomponent fiber” refers to a fiber that is similar to a bicomponent fiber but comprises three or more distinctly different polymeric components. For the purpose of this disclosure, any statement regarding bicomponent fibers should be reasonably understood to encompass multicomponent fibers as well.

“IR-emitting powder” refers to any material that is capable of absorbing ambient energy at limited or broad ranges of the spectrum, including but not limited to radiation in the microwave, infrared, visible, or ultraviolet ranges of the spectrum, then re-emitting some or all of that energy in the infrared range of the spectrum. IR-emitting materials can be mined as naturally-occurring minerals or can be synthesized, then manufactured into powders through pulverization, grinding, precipitation from solution, crystallization, or other such processes.

“Laminate” as a noun refers to a layered structure of sheet-like materials stacked and bonded so that the layers are substantially coextensive across the width of the narrowest sheet of material. The layers may comprise films, fabrics, or other materials in sheet form, or combinations thereof. For instance, a laminate may be a structure comprising a layer of film and a layer of fabric bonded together across their width such that the two layers remain bonded as a single sheet under normal use. A laminate may also be called a composite or a coated material. “Laminate” as a verb refers to the process by which such a layered structure is formed.

“Coextrusion” refers to a process of making multilayer polymer films. When a multilayer polymer film is made by a coextrusion process, each polymer or polymer blend comprising a layer of the film is melted by itself. The molten polymers may be layered inside the extrusion die, and the layers of molten polymer films are extruded from the die essentially simultaneously. In coextruded polymer films, the individual layers of the film are bonded together but remain essentially unmixed and distinct as layers within the film. This is contrasted with blended multicomponent films, where the polymer components are mixed to make an essentially homogeneous blend or heterogeneous mixture of polymers that are extruded in a single layer.

“Extrusion lamination” or “extrusion coating” refer to processes by which a film of molten polymer is extruded onto a solid substrate, in order to coat the substrate with the polymer film and to bond the substrate and film together.

“Stretchable” and “recoverable” are descriptive terms used to describe the elastomeric properties of a material. “Stretchable” means that the material can be extended by a pulling force to a specified dimension significantly greater than its initial dimension without breaking. For example, a material that is 10 cm long that can be extended to about 13 cm long without breaking under a pulling force could be described as stretchable. “Recoverable” means that a material which is extended by a pulling force to a certain dimension significantly greater than its initial dimension without breaking will return to its initial dimension or a specified dimension that is adequately close to the initial dimension when the pulling force is released. For example, a material that is 10 cm long that can be extended to about 13 cm long without breaking under a pulling force, and which returns to about 10 cm long or to a specified length that is adequately close to 10 cm could be described as recoverable.

“Elastomeric” or “elastomer” refer to polymer materials which can be stretched to at least about 150% of their original dimension, and which then recover to no more than 120% of their original dimension, in the direction of the applied stretching force. For example, an elastomeric film that is 10 cm long should stretch to at least about 15 cm under a stretching force, then retract to no more than about 12 cm when the stretching force is removed. Elastomeric materials are both stretchable and recoverable.

“Extensible” refers to polymer materials that can be stretched at least about 130% of their original dimension without breaking, but which either do not recover significantly or recover to greater than about 120% of their original dimension and therefore are not elastomeric as defined above. For example, an extensible film that is 10 cm long should stretch to at least about 13 cm under a stretching force, then either remain about 13 cm long or recover to a length more than about 12 cm when the stretching force is removed. Extensible materials are stretchable, but not recoverable.

“Brittle” refers to polymeric materials that are highly resistant to stretching and cannot be stretched more than 110% of their original dimension without breaking or cracking. For example, a brittle film that is 10 cm long cannot be stretched to more than about 11 cm under a stretching force without fracturing. Brittle films do not recover or recover only minimally when the stretching force is removed. Brittle materials are neither stretchable nor recoverable.

“Skin” or “skins,” when speaking of films, refer to thin outer layers of polymer film on one or both sides of another, central core of polymer film. For example, in the case of an AB or an ABA film structure, the A layers would be the skins.

“Core layer” or “core layers,” when speaking of films, refers to a thicker inner layer or layers of polymer film that are not the skins. For example, in an AB film where the B layer is thicker than the A layer, the B layer is the core layer. In an ABA film structure, the B layer is the core. In an ABCBA film structure, the B and C layers are all core layers.

“Activation” or “activating” refers to a process by which a film or fibrous material is stretched. “Effective emissivity” expresses the quantity of photons, specifically infrared photons in the 4-14 micron wavelength range of the spectrum that are emitted from the film, fabric, or laminate material, as a percentage of the total radiant energy impinging upon the material.

For the present invention, an IR-emitting powder is incorporated into a polymeric matrix to form polymeric materials such as polymeric fibers or films. The IR-emitting powder of the present invention should comprise a material with an IR emissivity of at least about 50%, preferably at least about 65%, more preferably at least about 75%, more preferably at least about 85%. IR-emitting powders can comprise many inorganic and organic materials. For instance, many metal oxides can be used as IR-emitting powders. Examples of such metal oxides include, but are not limited to, alumina (Al₂0₃), magnesia (MgO), zirconia (ZrOr), titanium dioxide (TiO₂), silicon dioxide (SiOr), chromium oxide (Cr₂O₃), ferrite (FeO₂, Fe₃O₄), spinel (MgOAl₂O₃), barium oxide (BaO), zinc oxide (ZnO), tin oxide (SnO₂), and tungsten trioxide (WO₃). Crystalline minerals, including but not limited to mica, calcite, rock crystal and tourmaline, can be used as IR-emitting powders. In particular, tourmaline, a boron silicate mineral with complex chemical structures, is a mineral oxide with favorable IR-emitting characteristics. Non-oxide ceramics, including but not limited to boron carbide (B₄C), silicon carbide (SiC), titanium carbide (TiC), molybdenum carbide (MoC), tungsten carbide (WC), boron nitride (BN), aluminum nitride (AlN), silicon nitride (Si₃N₄) and zirconium nitride (ZrN), can also be IR-emitting materials. Non-metallic IR-emitting materials include graphite, carbon black and charcoal. Metals and metal alloys, including but not limited to tungsten, molybdenum, vanadium, platinum, nickel, copper, nichrome, stainless steel, and alumel, can also be used as IR-emitting powders. Combinations, mixtures, or blends of the IR-emitting materials described herein are also contemplated as embodiments of the present invention.

As previously stated, the IR-emitting material must be a fine powder for the purposes of the present invention. Suitable particle sizes for these powders include particles ranging from about 10 nm to about 10 μm across.

The polymers used as the matrix materials for the present invention include any extrudable thermoplastic polymer. Suitable polymers for the polymeric matrix materials include, but are not limited to, polyolefins, for example, polyethylene homopolymers and copolymers, and polypropylene homopolymers and copolymers, functionalized polyolefins, polyesters, poly(ethylene oxides), poly(ester-ethers), polyamides, including nylons, poly(ether-amides), polyacrylates, polyacrylonitrile, polyvinyl chloride, polyether sulfones, fluoropolymers, polyurethanes, styrenic block copolymers, and the like. Polyethylene homopolymers include those of low, medium or high density and/or those formed by high pressure or low pressure polymerization. Polyethylene and polypropylene copolymers include, but are not limited to, copolymers with C₄-C₈ alpha-olefin monomers, including 1-octene, 1-butene, 1-hexene and 4-methyl pentene. The polyethylene may be substantially linear or branched, and may be formed by various processes known in the art using catalysts such as Ziegler-Natta catalysts, metallocene or single-site catalysts or others widely known in the art. Examples of suitable copolymers include, but are not limited to, copolymers such as poly(ethylene-butene), poly(ethylene-hexene), poly(ethylene-octene), and poly(ethylene-propylene), poly(ethylene-vinylacetate), poly(ethylene-methylacrylate), poly(ethylene-acrylic acid), poly(ethylene-butylacrylate), poly(ethylene-propylenediene), and/or polyolefin terpolymers thereof. Suitable polyesters include polyethylene terephthalate. Suitable polyamides include nylon 6, nylon 6,6, and nylon 6,12. Styrenic block copolymers include styrene-butadiene-styrene (SBS), styrene-isoprene-styrene (SIS), styrene-ethylenebutylene-styrene (SEBS), styrene-ethylenepropylene-styrene (SEPS), and other similar polymers.

One problem with manufacturing polymeric fibrous materials containing IR-emitting powders is that the powders can make the polymeric fibers hard to spin. If the powder content of the fibers is very high, such as greater than about 1% to 5% of the total fiber mass, it can become very difficult to spin the fibers using conventional fiber-spinning technology. Additionally, in the past it has been thought that high concentrations of IR-emitting powders were too abrasive to be incorporated into fibers. The concern was that high loadings of the IR-emitting powders would quickly damage the fiber-forming equipment because of abrasion from the powders.

IR-emitting powders can be incorporated into the sheath of sheath-core bicomponent fibers at much higher concentrations of powder than were previously thought possible. It is possible to incorporate IR-emitting powder comprising a concentration of about 1% up to about 10%, up to about 20%, up to about 25%, up to about 30%, or up to about 50% of powder by weight in the sheath component of the fiber, and still easily draw fibers, as long as the core component of the bicomponent fibers comprises at least about 50% or more of the total fiber volume.

The IR-emitting powder can also be incorporated into one or more components of other bicomponent or multicomponent fibers. For instance, the IR-emitting powder may be incorporated into one or more layers of a multilayer sheath-core fiber construction, as illustrated in FIG. 1a. Alternatively, the IR-emitting powder may be incorporating into the islands component of an islands-in-the-sea bicomponent or multicomponent fiber, as illustrated in FIG. 1b. Alternatively, IR-emitting powders may be incorporated into the wedges or walls of a bicomponent or multicomponent fiber with a “pie” or “orange” cross-section, as illustrated in FIG. 1c.

For bicomponent or multicomponent fibers, the fiber component containing the IR-emitting powder can constitute up to about 50% of the total bicomponent fiber structure, with the other fiber component(s) constituting the remaining percentage of the total fiber structure. More preferably, the IR-emitting component can constitute up to about 30%. up to about 25%, up to about 20%, up to about 15%, or up to about 10% of the total fiber structure, with the other fiber component(s) constituting the remaining percentage of the total fiber structure.

Increasing the surface area of the polymeric fibers can increase the IR emissivity of the material. By increasing the active surface area of the fiber, there is more area to capture and absorb impinging radiation, thereby increasing the opportunity for the IR-emitting powder particles to encounter and absorb the impinging radiation, then convert and re-emit that energy as photons in the IR spectrum. The IR-emitting powder may be incorporated throughout the fibers with increased surface area, or the powder may be incorporated into one or more components of a bicomponent or multicomponent fiber structure with increased surface area.

The surface area of a polymeric fiber can be increased in many ways. One simple way to do this is to spin fibers with non-circular cross sections. Such fibers can be made with oblong, dog-bone, trilobal, multilobal, star, or other non-circular shapes. FIGS. 2 a-e illustrate a few of the many possible shapes with increased surface areas.

One possible embodiment of the present invention is to create bicomponent multilobal fibers that are extruded such that the IR-emitting powders are concentrated in the lobe areas. An example of this embodiment is illustrated in FIG. 3. This embodiment increases the surface area of the fiber, exposes the IR-emitting material on the surface, but maintains the strength and toughness of the unfilled central region of the fiber.

Another way to increase the surface area of a polymeric fiber is to incorporate a foaming agent into the fiber matrix. The foaming agent can be incorporated throughout the entire fiber or, preferably, only in one component of a bicomponent or multicomponent fiber. The foaming agent creates tiny open or closed cells within the polymer matrix of the fiber, which results in a dramatic increase in the surface areas within the fiber. These surface areas allow impinging radiation to scatter and, essentially, “bounce” around the interior of the fiber until the radiation either escapes or is absorbed by the IR-emitting powder.

Yet another way to increase the surface area of a polymeric fiber is to microtexture the surface. For a sheath-core bicomponent fiber, if the core material is somewhat more elastic and has more stretch-and-recover properties than the sheath, the fibers can be stretched to some degree and then recover to roughly the original length of the fiber. If the sheath of the bicomponent fiber is extensible but less elastic than the core material, though, the sheath will stretch when the fiber is stretched, but the sheath will not recover when the stretching force is relaxed. Instead, the sheath material will tend to wrinkle or pucker over the surface of the fiber. This wrinkling is also called microtexturing, due to the fact that the individual wrinkles are often too small to be visible to the naked eye. Instead, the eye merely perceives a matte surface somewhat resembling an embossed surface. FIG. 4 illustrates a microtextured fiber of the present invention. Note that, for fibers with microtextured surfaces, it is not necessary that the core polymer composition be truly elastomeric as defined above. Indeed, the core polymer composition need only be extensible. It is only necessary that the core polymer composition have greater recovery than the sheath polymer composition after the fiber is stretched.

Yet another way to increase the surface area of a polymeric fiber is to form the fiber with a sheath of a brittle polymer composition, then stretch the fiber to crack the brittle polymer sheath. For this embodiment, the brittle polymer in the sheath of the fiber may comprise any common extrudable, brittle polymer known in the art, such as polystyrene, polymethylmethacrylate, other acrylate polymers, polyesters, polycarbonates, etc. One brittle polymer that is particularly preferred is polystyrene. For this embodiment, the polymer composition of the fiber core should comprise an extensible or elastomeric polymer. This will give the overall fiber the toughness to be stretched, in order to crack the brittle polymeric sheath, without breaking the entire fiber. FIG. 5 illustrates a bicomponent fiber with a brittle sheath as one embodiment of the invention.

Yet another method of increasing the surface area of the polymeric fibers is to extrude bicomponent fibers which can then be separated or split into microfibers, using known microfiber-manufacturing technology. FIGS. 6a and 6b illustrate examples of splittable bicomponent fibers before and after they are separated into microfibers. The first unfilled component of the original bicomponent fiber acts as a carrier matrix that makes it possible to spin the pre-split fiber, and the second component containing the IR-emitting powders is then split into the microfibers, which dramatically increases the available surface area of these fibers.

Yet another method of increasing the surface area of the polymeric fibers is to etch the surface of the fiber, using an etching technique such as chemical etching or plasma etching. Etching attacks the surface of the fibers and preferentially removes areas of less polymer crystallinity. This both erodes the surface, thereby creating a more rugged surface with increased area, and exposes the more crystalline polymer structure within the fiber.

Yet another method of increasing the surface area of the polymeric fibers is to emboss the surface of the fiber. Some continuous fiber manufacturing techniques, such as cold drawing or mechanical drawing, involve drawing the extruded semimolten fiber from the spinneret using a stripper roll rather than simply laying the extruded fiber on a forming surface. If the stripper roll is engraved with an embossing pattern, the embossing pattern will be transferred to the drawn fiber.

Once the inventive IR-emitting fibers are formed, they may be further processed into fabrics or other fibrous webs. These fabrics or other fibrous webs may be made entirely of the inventive fibers, or the inventive fibers may be mixed with traditional fibers in order to create fabrics with the desired physical and aesthetic properties, For durable goods, the inventive fibers can be woven or knitted into fabrics that can then be used to fashion garments, protective outer wear, blankets, or other such end-use products. For limited-use or disposable goods, it may be desirable to form nonwoven fabrics from the inventive fibers. Methods of making woven, knitted, and nonwoven fabrics are well known in the art.

Another embodiment of the present invention are polymeric films that comprise IR-emitting powders. Films, like fibers, may comprise similar IR-emitting powders and polymeric matrix components. Extrudable thermoplastic polymeric materials suitable for such polymeric film materials are known.

However, the inventor has discovered that IR-emitting powders can be incorporated into a thin skin layer on a multilayer film and obtain acceptable emissivity results. By filling the skin layer of the film with IR-emitting powders and having a core layer with little or no IR-emitting powder, it is possible to manufacture thin films that are not brittle and less expensive than films that contain the IR-emitting powder throughout the film structure. Additionally, the skin layer may contain a much higher loading of IR-emitting powder, and still be easily processed into a flexible, tough film. It is possible to incorporate IR-emitting powder comprising a concentration of about 1% up to about 10%, up to about 20%, up to about 25%, up to about 30%, up to about 50%, or up to about 80% of powder by weight in the skin layer of the film. The film skin layer can constitute up to about 30% of the total film structure, with the other film layer(s) constituting the remaining percentage of the total film structure. More preferably, the skin layer can constitute up to about 25%, up to about 20%, up to about 15%, up to about 10%, or up to about 5% of the total film structure, with the other film layer(s) constituting the remaining percentage of the total film structure.

Any film-forming process can prepare the inventive multilayer IR-emitting film. In a specific embodiment, a coextrusion process, such as cast coextrusion or blown-film coextrusion, is used to form the multilayer film. Coextrusion of multilayer films by cast or blown processes are well known.

In another embodiment, the inventor has discovered that a multilayer film may be made which comprises one skin layer filled with IR-emitting powder, a core layer comprising an unfilled polymer, and another skin layer that comprises a light-reflective treatment, coating, or material such as a metalized particles, metalized film or a thin metal foil. The reflective skin layer may enhance the effective emissivity of the IR-emitting component of the film. Impinging photons that are not absorbed by the IR-emitting powder as they pass through the film would be reflected back through the film and thereby have another chance to be absorbed by the IR-emitting material. As photons are emitted from the IR-emitting component, the reflective skin layer will also reflect these emitted photons in the desired direction. Thus the effective emissivity of the IR-emitting material is improved.

Further increasing the surface area of the polymeric film can increase the IR emissivity of the inventive material. Just as with fibers, by increasing the surface area of the film, there is more area to capture impinging radiation, thereby increasing the opportunity for the IR-emitting powder particles to encounter and absorb the impinging radiation, then convert and re-emit that energy as photons with wavelengths in the IR spectrum. The IR-emitting powder may be incorporated throughout the films with increased surface area, or the powder may be incorporated into one or more layers, preferably the skin layer, of a multilayer film with increased surface area.

One method of increasing the surface area of the polymeric film is to emboss the surface of the film. In a cast extrusion process, the molten or semi-molten polymer web is cast onto a chilled cast roll, where the film is rapidly quenched and solidified. If the cast roll is engraved with an embossing pattern, the embossing pattern will be transferred to the extruded film.

Another way to increase the surface area of a polymeric film is to incorporate a foaming agent into the film composition. The foaming agent can be incorporated throughout the entire film or only in the skin layer of a multilayer film. The foaming agent creates tiny open or closed cells within the polymer matrix of the film, which results in a dramatic increase in the surface areas within the film. These surface areas allow impinging radiation to scatter and, essentially, “bounce” around the interior of the film until the radiation either escapes or is absorbed by the IR-emitting powder.

Yet another way to increase the surface area of a polymeric film is to microtexture the surface. For a multilayer film, if the core layer material is somewhat elastic and has stretch-and-recover properties, the film can be stretched to some degree and then recover to roughly the original length of the film. Just as with fibers, if the skin of the multilayer film is extensible but less elastic than the core layer the skin will stretch when the film is stretched, but the skin layer will not recover when the stretching force is relaxed. Instead, the skin layer will tend to wrinkle or pucker over the surface of the film. This wrinkling is also called microtexturing. Note that, for films with microtextured surfaces, it is not necessary that the core layer polymer composition be truly elastomeric as defined above. Indeed, the core layer polymer composition need only be extensible. It is only necessary that the core layer polymer composition have greater recovery than the skin layer polymer composition after the film is stretched.

Yet another way to increase the surface area of a polymeric film is to extrude a multilayer film with a brittle polymer skin, then stretch the film to crack the brittle skin. For this embodiment, the brittle polymer comprising the skin of the film may comprise any common extrudable, brittle polymer known in the art, such as polystyrene, polymethylmethacrylate, other acrylate polymers, polyesters, polycarbonates, etc. One brittle polymer that is particularly preferred is polystyrene. For this embodiment, the polymer composition of the film core layer should comprise an extensible or elastomeric polymer. This will give the overall film the toughness to be stretched, in order to crack the brittle polymeric skin, without breaking the entire film.

Yet another method of increasing the surface area of the polymeric films is to etch the surface of the film, using an etching technique such as chemical etching or plasma etching. Etching attacks the surface of the film and preferentially removes areas of less polymer crystallinity. This both erodes the surface, thereby creating a more rugged surface with increased area, and exposes the more crystalline polymer structure within the film.

For some embodiments of the invention, whether for fibers, fibrous webs or films, it is necessary to stretch or activate the inventive material in order to increase the surface area of the material. Stretching is necessary in order to create microtextured fibers or film, and stretching is also necessary in order to break or crack the brittle polymer sheath or skin layer on the fibers or film. The inventive material can be activated in a number of ways. For instance, the material can be stretched, folded, corrugated, calendered with a patterned roll, or otherwise deformed in such a way that the core layer is extended and the skin layer is either extended or broken. A preferred means of stretching the material is by known stretching techniques, such as machine-direction orientation (MIX)), teetering, or incremental stretching. A particularly preferred method of activating the material is by incrementally stretching the material between intermeshing rollers, as described in U.S. Pat. No. 4,144,008.

It is to be understood that additional processing steps such as printing or slitting the inventive material, laminating additional layers onto the inventive material, and other such processes may be added and are within the scope of this invention.

The inventive material, particularly fibrous webs or films, may be laminated to a substrate layer by known lamination means. The substrate layer can be any extensible sheet-like material, such as another fabric, another polymer film, or paper. A particularly useful material is an IR-emitting material comprising both an IR-emitting fibrous web and an IR-emitting film.

The inventive material may be laminated to the substrate layer by known lamination means. These lamination means include extrusion lamination, adhesive lamination, thermal bonding, ultrasonic bonding, calender bonding, and other such means. Combinations of these bonding methods are also within the scope of the present invention.

The inventive material can be laminated to one or more substrate layers at any point in the process. Specifically, the inventive material can be laminated to a substrate layer before or after the material is activated.

The following examples are presented to illustrate diverse aspects of the present invention. These examples are not intended to limit the invention in any way.

### Example 1

Bicomponent fibers of sheath/core construction were manufactured according to the present invention. The sheath of the fibers comprised an IR-emitting powder masterbatch of nano-sized particles of bamboo charcoal, 20% by weight, in a polypropylene matrix, sold as Product Code ZT-MB020 by Shanghai Huzheng Nano Technology Ltd., Shanghai, China. The core of the bicomponent fibers comprised polypropylene, PRO-FAX® PH835 from LyondellBasell, Houston, Tex., with an MFI of 35. The structure of the fibers was 10% sheath and 90% core by volume. The fibers were successfully spun into good fibers that were formed into a nonwoven web.

### Example 2

Bicomponent fibers were manufactured that had the same composition as Example 1, but the sheath polymer composition contained only 10% by weight of the IR-emitting powder masterbatch. The structure of the fibers was 25% sheath and 75% core by volume. The fibers were successfully spun into good fibers that were formed into a nonwoven web.

### Comparative Example 1

Bicomponent fibers of sheath/core construction were manufactured according to the present invention. The sheath of the fibers comprised an IR-emitting powder masterbatch of nano-sized particles of bamboo charcoal, 20% by weight, in a polypropylene matrix, sold as Product Code ZT-MB020 by Shanghai Huzheng Nano Technology Ltd., Shanghai, China. The core of the bicomponent fibers comprised polypropylene, PRO-FAX® PH835 from LyondellBasell, Houston, Tex., with an MFI of 35. The structure of the fibers was 25% sheath and 75% core by volume. These fibers could not be successfully spun into good fibers, which broke during the drawing process and could not be formed into a web, and the experiment had to be aborted.

Examples 1 and 2 demonstrate that bicomponent fibers containing high concentrations of IR-emitting powders can be successfully spun, so long as the underlying core component of the fiber constitutes a sufficient fraction of the overall structure. In contrast, Comparative Example 1 shows that if the core component of the fiber is less than a sufficient fraction of the overall structure, the bicomponent fibers containing high concentrations of IR-emitting powders cannot be successfully manufactured. It is anticipated that the optimum or a preferred sheath-to-core ratio for a given fiber composition will vary depending on a number of factors, including but not limited to the physical properties of the supporting polymer matrix and the identity, concentration, and particle size distribution of the IR-emitting powder. Determining a preferred or optimum sheath-to-core ratio for bicomponent fibers of the present invention should be a matter of routine experimentation for one skilled in the art.

### Example 3

A foamed polymer film was prepared comprising about 48.5% Vistamaxx 6102 polyolefin, 3% Ecocell foaming agent, and 48.5% IR-emitting masterbatch containing 20% solids in a polyethylene matrix, sold as Product Code ARP-MB020 by Shanghai Huzheng Nano Technology Ltd., Shanghai, China. The final film formulation contained about 10% IR-emitting powder. A comparable film containing the same components except the Ecocell foaming agent was also prepared. The foamed film and flat film were tested for emissivity by CI Systems, Simi Valley, Calif.

FIGS. 7a and 7b show the emissivity scans for the foamed film and the flat film, respectively. The testing showed that the foamed film emissivity remained between 80 and 100% for the entire 4 to 14 micron wavelength range, which is the beneficial wavelength range of the IR spectrum. The emissivity of the flat film, in contrast, was under 80% for much of this wavelength range. The foamed film thus shows superior emissivity when compared to the flat film.

This has been a description of the present invention along with the preferred method of practicing the present invention. However, the invention itself should only be defined by the appended claims, WHEREIN WE CLAIM:

What is claimed is:

1. An IR-emitting bicomponent polymeric fiber comprising
a) a first polymer composition comprising finely-divided particles of an IR-emitting material and a thermoplastic polymer, and
b) a second polymer composition comprising a thermoplastic polymer,
wherein the first polymer composition comprises the sheath of a sheath-core bicomponent fiber structure and the second polymer composition comprises the core of said sheath-core bicomponent fiber structure.

2. The IR-emitting bicomponent polymeric fiber according to claim 1, wherein the IR-emitting material comprises about 1-50% of the first polymer composition.

3. The IR-emitting bicomponent polymeric fiber according to claim 1, wherein the IR-emitting material comprises about 1-25% of the first polymer composition.

4. The IR-emitting bicomponent polymeric fiber according to claim 1, wherein the IR-emitting material comprises about 10-25% of the first polymer composition.

5. The IR-emitting bicomponent polymeric fiber according to claim 1, wherein the sheath to core ratio within said bicomponent fiber is in the range of about 1%/99% to about 50%/50%, respectively.

6. The IR-emitting bicomponent polymeric fiber according to claim 1, having a sheath to core ratio within said bicomponent fiber in the range of about 10%/90% to about 50%/50%, respectively.

7. The IR-emitting bicomponent polymeric fiber according to claim 1, having a sheath to core ratio within said bicomponent fiber is in the range of about 10%/90% to about 25%/75%, respectively.

8. The IR-emitting bicomponent polymeric fiber according to claim 1, wherein the IR-emitting material is selected from the group consisting of metal oxides, crystalline minerals, ceramic carbides, ceramic nitrides, metals, metal alloys, carbon, and combinations thereof.

9. The IR-emitting bicomponent polymeric fiber according to claim 8, wherein the IR-emitting material is selected from the group consisting of alumina, magnesia, zirconia, titanium dioxide, silicon dioxide, chromium oxide, ferrite, spinel, barium oxide, zinc oxide, tin oxide, tungsten trioxide, mica, calcite, rock crystal, tourmaline, boron carbide, silicon carbide, titanium carbide, molybdenum carbide, tungsten carbide, boron nitride, aluminum nitride, silicon nitride, zirconium nitride, graphite, carbon black, charcoal, tungsten, molybdenum, vanadium, platinum, nickel, copper, nichrome, stainless steel, alumel, and combinations thereof.

10. The IR-emitting bicomponent polymeric fiber according to claim 1, wherein the finely-divided particles of the IR-emitting material are from about 10 nm to about 10 μm in size.

11. The IR-emitting bicomponent polymeric fiber according to claim 1, wherein the polymer in the first polymer composition and the polymer in the second polymer composition are each selected from the group consisting of polyolefins, polyesters, polyamides, functionalized polyolefins, poly(ethylene oxides), poly(ester-ethers), poly(ether-amides), polyacrylates, cellulosic polymers, polyaramides, polyvinyl chloride, polyether sulfones, fluoropolymers, polyurethanes, styrenic block copolymers, copolymers thereof and blends thereof.

12. The IR-emitting bicomponent polymeric fiber according to claim 1, wherein the polymer in the first polymer composition and the polymer in the second polymer composition are each selected from the group consisting of polyethylene; polypropylene; polyethylene terephthalate; nylon 6; nylon 6,6; nylon 6,12; polyacrylonitrile; rayon; polyvinyl chloride, styrene-butadiene-styrene; styrene-isoprene-styrene; styrene-ethylenebutylene-styrene; copolymers thereof; and blends thereof.

13. The IR-emitting bicomponent polymeric fiber according to claim 1, wherein the polymer in the first polymer composition and the polymer in the second polymer composition are the same polymer.

14. The IR-emitting bicomponent polymeric fiber according to claim 1, wherein the polymer in the first polymer composition and the polymer in the second polymer composition are different polymers.

15. The IR-emitting bicomponent polymeric fiber according to claim 1, formed into a fibrous web.

16. The fibrous web according to claim 15, wherein said fibrous web comprises a woven, knitted, or nonwoven fabric.

17. The fibrous web according to claim 15, wherein said fibrous web also comprises traditional fibers.

18. The fibrous web according to claim 15, wherein said fibrous web is bonded to a substrate layer to form a laminated structure.

19. The laminated structure according to claim 18, wherein the fibrous web is a nonwoven fabric and the substrate layer is a polymeric film.

20. An IR-emitting polymeric fiber comprising a first polymer composition comprising finely-divided particles of an IR-emitting material and a thermoplastic polymeric matrix, wherein the fiber surface area is greater than the surface area of a comparable fiber of circular cross-section with a smooth surface.

21. An IR-emitting polymeric fiber according to claim 20, wherein the fiber is a bicomponent fiber comprising
a) said first polymer composition comprising finely-divided particles of an (R-emitting material and a thermoplastic polymeric matrix, and
b) a second polymer composition comprising a thermoplastic polymer.

22. An IR-emitting polymeric fiber according to claim 20, wherein the fiber has a non-circular shaped cross section.

23. An IR-emitting polymeric fiber according to claim 22, wherein the fiber has an oblong, dog-bone, trilobal, multilobal, or star shaped cross section.

24. An IR-emitting bicomponent polymeric fiber according to claim 21, wherein the fiber has a non-circular shaped cross section.

25. An IR-emitting bicomponent polymeric fiber according to claim 24, wherein the fiber has an oblong, dog-bone, trilobal, multilobal, or star shaped cross section.

26. An IR-emitting bicomponent polymeric fiber according to claim 25, wherein said first polymer composition comprises all or part of the surface of the fiber.

27. An IR-emitting bicomponent polymeric fiber according to claim 25, wherein
a) the fiber is trilobal or multilobal in shape, and
b) the first polymer composition is located substantially in lobed areas of the fiber, and
c) the second polymer composition is located substantially in a central area connecting the lobed areas of the fiber.

28. An IR-emitting polymeric fiber according to claim 20, wherein the first polymer composition comprises a foamed polymer.

29. An IR-emitting bicomponent polymeric fiber according to claim 21, wherein the first polymer composition comprises a foamed polymer and the first polymer composition comprises the sheath component of a sheath-core structure.

30. An IR-emitting polymeric fiber according to claim 20 wherein the surface of the fiber is microtextured to increase surface area.

31. An IR-emitting bicomponent polymeric fiber according to claim 21, wherein the bicomponent fiber comprises a sheath-core structure and the surface of the sheath layer is microtextured to increase the fiber surface area.

32. An IR-emitting bicomponent polymeric fiber according to claim 31, wherein the surface of the sheath layer is microtextured by stretching.

33. An IR-emitting bicomponent polymeric fiber according to claim 31, wherein the surface of the sheath layer is microtextured by incremental stretching.

34. An IR-emitting bicomponent polymeric fiber according to claim 21, wherein the bicomponent fiber comprises a sheath-core structure and the sheath layer comprises a brittle polymer which is cracked to increase the fiber surface area.

35. An IR-emitting bicomponent polymeric fiber according to claim 34, wherein the brittle polymer comprises polystyrene, polymethylmethacrylate, polyester, polycarbonate, copolymers thereof, or blends thereof.

36. An IR-emitting bicomponent polymeric fiber according to claim 34, wherein the brittle polymer is cracked by stretching, folding, corrugating, calendering, or combinations thereof.

37. An IR-emitting bicomponent polymeric fiber according to claim 36, wherein the brittle polymer is cracked by incremental stretching.

38. An IR-emitting bicomponent polymeric fiber according to claim 21, wherein the fiber is split to form microfibers thereby increasing a surface area and wherein said micro fibers comprise the first polymeric composition.

39. An IR-emitting bicomponent polymeric fiber according to claim 38, wherein the splittable bicomponent fiber has an orange or islands-in-the-sea structure.

40. An IR-emitting bicomponent polymeric fiber according to claim 20, wherein the fiber is embossed to increase the fiber surface area.

41. An IR-emitting bicomponent polymeric fiber according to claim 20, wherein the fiber is etched to increase the fiber surface area.

42. The IR-emitting polymeric fiber according to claim 20, formed into a fibrous web.

43. The fibrous web according to claim 42, wherein said fibrous web comprises a woven, knitted, or nonwoven fabric.

44. The fibrous web according to claim 42, wherein said fibrous web also comprises traditional fibers.

45. The fibrous web according to claim 42, wherein said fibrous web is bonded to a substrate layer to form a laminated structure.

46. The laminated structure according to claim 45, wherein the fibrous web is a nonwoven fabric and the substrate layer is a polymeric film.

47. An IR-emitting multilayer polymeric film comprising
a) a first polymer composition comprising finely-divided particles of an IR-emitting material and a thermoplastic polymeric matrix, and
b) a second polymer composition comprising a thermoplastic polymer,
wherein the first polymer composition comprises the skin layer of the multilayer film and the second polymer composition comprises the core layer of the multilayer film.

48. The IR-emitting multilayer polymeric film according to claim 47, wherein the IR-emitting material comprises about 1-50% of the first polymer composition.

49. The IR-emitting multilayer polymeric film according to claim 47, wherein the IR-emitting material comprises about 1-25% of the first polymer composition.

50. The IR-emitting multilayer polymeric film according to claim 47, wherein the IR-emitting material comprises about 10-25% of the first polymer composition.

51. The IR-emitting multilayer polymeric film according to claim 47, wherein the IR-emitting material is selected from the group consisting of metal oxides, crystalline minerals, ceramic carbides, ceramic nitrides, metals, metal alloys, carbon, and combinations thereof.

52. The IR-emitting multilayer polymeric film according to claim 51, wherein the IR-emitting material is selected from the group consisting of alumina, magnesia, zirconia, titanium dioxide, silicon dioxide, chromium oxide, ferrite, spinel, barium oxide, zinc oxide, tin oxide, tungsten trioxide, mica, calcite, rock crystal, tourmaline, boron carbide, silicon carbide, titanium carbide, molybdenum carbide, tungsten carbide, boron nitride, aluminum nitride, silicon nitride, zirconium nitride, graphite, carbon black, charcoal, tungsten, molybdenum, vanadium, platinum, nickel, copper, nichrome, stainless steel, alumel, and combinations thereof.

53. The IR-emitting multilayer polymeric film according to claim 47, wherein the finely-divided particles of the IR-emitting material are range from about 10 nm to about 10 μm in size.

54. The IR-emitting multilayer polymeric film according to claim 47, wherein the thermoplastic polymeric matrix in the first polymer composition and the polymer in the second polymer composition are each selected from the group consisting of polyolefins, polyesters, polyamides, functionalized polyolefins, poly(ethylene oxides), poly(ester-ethers), poly(ether-amides), polyacrylates, polyvinyl chloride, polyether sulfones, fluoropolymers, polyurethanes, polyolefinic elastomers, styrenic block copolymers, copolymers thereof and blends thereof.

55. The IR-emitting multilayer polymeric film according to claim 47, wherein the thermoplastic polymeric matrix in the first polymer composition comprises polyethylene, polypropylene, polyethylene terephthalate, nylon 6, nylon 6,6, copolymers thereof or blends thereof.

56. The IR-emitting multilayer polymeric film according to claim 47, having a film skin surface area greater than a surface area of a comparable film with a smooth surface.

57. The IR-emitting multilayer polymeric film according to claim 56, wherein the film is embossed to increase the film surface area.

58. The IR-emitting multilayer polymeric film according to claim 56, wherein the first polymer composition of the film comprises a foamed polymer to increase the film surface area.

59. The IR-emitting multilayer polymeric film according to claim 56, wherein the skin layer of the film is microtextured to increase the film surface area.

60. The IR-emitting multilayer polymeric film according to claim 59, wherein the skin layer of the film is microtextured by stretching.

61. The IR-emitting multilayer polymeric film according to claim 59, wherein the skin layer of the film is microtextured by incremental stretching.

62. The IR-emitting multilayer polymeric film according to claim 56, wherein the first polymer composition of the film comprises a brittle polymer skin layer which is cracked to increase a film surface area.

63. The IR-emitting multilayer polymeric film according to claim 62, wherein the brittle skin layer of the film is cracked by stretching, folding, corrugating, or calendering.

64. The IR-emitting multilayer polymeric film according to claim 62, wherein the brittle skin layer of the film is cracked by incremental stretching.

65. The IR-emitting multilayer polymeric film according to claim 47, comprising a second skin layer comprising a reflective material.

66. The IR-emitting multilayer polymeric film according to claim 65, wherein the reflective material is a metalized film, metalized particles, or a metal foil.

67. The IR-emitting multilayer polymeric film according to claim 47, wherein said film is bonded to a substrate layer to form a laminated structure.

68. The film according to claim 67, wherein the substrate layer is a fibrous web.

69. The laminated structure according to claim 68, wherein the substrate layer is a nonwoven fabric.

70. A laminated material comprising:
a) a layer of an IR-emitting polymeric fibrous web of fibers comprising a polymer composition comprising finely-divided particles of an IR-emitting material and a first thermoplastic polymeric matrix, and
b) a layer of an IR-emitting polymeric film comprising a polymer composition comprising finely-divided particles of an IR-emitting material and a second thermoplastic polymeric matrix.

71. A laminated material according to claim 70, wherein the fibrous web is a nonwoven fabric.
